(19) United States
(12) Patent Application Publication
(10) Pub. No.: US 2011/0077585 A1
(43) Pub. Date: Mar. 31, 2011

(54) APC Dual Mode Theraputic Balloon Dilator

(76) Inventor: Jerome Canady, Lakeland, FL (US)

(21) Appl. No.: 12/964,272

(22) Filed: Dec. 9, 2010

Related U.S. Application Data
(63) Continuation of application No. 11/347,247, filed on Feb. 6, 2006, now abandoned.
(60) Provisional application No. 60/650,657, filed on Feb. 7, 2005.

(51) Int. Cl. A61M 37/00; A61M 25/00; A61B 18/18

(52) U.S. Cl. 604/23; 604/528; 606/49

(57) ABSTRACT

A multi-mode therapeutic balloon dilator capable of stent placement, argon plasma coagulation (APC) and monopolar electrosurgery. The device comprises a housing or base having a mechanism for moving a wire within an elongated flexible tube, a balloon port, an injection portion, a monopolar connector, and an argon gas connector.

### CROSS-REFERENCE TO RELATED APPLICATIONS

This application is a continuation of U.S. Nonprovisional patent application Ser. No. 11/347,247 entitled “APC Dual Mode Therapeutic Balloon Dilator” and filed on Feb. 6, 2006, which claims the benefit of the filing date of U.S. Provisional Application Ser. No. 60/650,657 entitled “APC Dual Mode Therapeutic Balloon Dilator,” and filed on Feb. 7, 2005 by inventor Jerome Canady.

The above cross-referenced related applications are hereby incorporated by reference herein in their entirety.

### STATEMENT REGARDING FEDERALLY SPONSORED RESEARCH OR DEVELOPMENT

Not applicable.

### BACKGROUND OF THE INVENTION

1. Field of the Invention

The present invention relates to surgical device capable of performing argon plasma coagulation after stent placement.

2. Brief Description of the Related Art

Controlling or arresting blood loss is of high priority during surgery so as to avoid or minimize the necessity of introducing foreign blood or blood products into a patient. This has increased in importance due to concern over contamination of the blood supply by viral agents which cause, for example, acquired immune deficiency syndrome (AIDS), hepatitis, and the like.

Standard means for controlling traumatic and surgical blood loss are electrosurgical generators and lasers, which respectively direct high-frequency electrical currents or light energy to localize heat in bleeding vessels so as to coagulate the overlying blood and vessel walls.

Argon beam coagulators additionally have been demonstrated to be effective tissue coagulators. Examples of argon beam coagulators for use in open surgery can be found in U.S. Pat. Nos. 4,040,426 to Morrison and 4,781,175 to McGreevy. Argon beam coagulators for use rigid and flexible endoscopy also are known. An example of a device for flexible endoscopy may be seen in U.S. Pat. No. 5,207,675 to the present inventor. In some embodiments in that patent, the inventor disclosed dual modality devices that could be used either for argon plasma coagulation or for traditionally electrocautery in an endoscopic environment. The inventor also disclosed an embodiment having the dual modality of argon plasma coagulation and endoscopic biopsy forceps. In that embodiment, argon plasma coagulation could be used by a surgeon while the biopsy forceps were withdrawn inside the flexible endoscopic tube. The biopsy forceps could then be extended and used, but argon plasma coagulation was not performed with the biopsy forceps extended from the end of the tube.

Balloon dilators of various types are known. Such devices are disclosed, for example, in U.S. Pat. No. 6,974,441 entitled “Inflatable Intraluminal Molding Device,” and U.S. Pat. No. 5,951,514.

### SUMMARY OF THE INVENTION

A combination argon plasma coagulation and balloon dilator device and method are disclosed. The APC Dual Mode Therapeutic balloon dilator of the present invention is designed for use in argon plasma coagulation for high grade dysplasia, tumors, strictures and stenosis of lumens after stent placement. The multipurpose probe will also have the ability to pass a guide wire through a tight stricture, irrigation and injection port, and use conventional cautery and cutting. The therapeutic balloon can be used for dilation or designed for endoscopic positioning for delivery of argon plasma.

In a first embodiment of the present invention, a surgical device comprises an elongate tube having a proximal end and a distal end and having a first and second channels therein each having a proximal end and a distal end and the first channel having a port in a distal end region of the tube. A wire having a proximal end and a distal end is within the first channel. A housing is connected near the proximal end of the tube. The housing comprises means for connecting the proximal end of the wire to a source of RF energy and means for connecting the proximal end of the first channel to a source of an inert ionizable gas. An inflatable member is connected to the tube. The inflatable member is inflatable through the second channel in the tube.

The surgical device may have a third channel therein, for example, for inserting various surgical tools. The surgical device may be designed for laparoscopic, endoscopic, or open surgery or other types of surgery. In other words, the surgical device of present invention may be designed to be inserted into any type of scope, such as a laparoscope or an endoscope. The tube may be rigid, flexible, or semi-rigid. The inert ionizable gas may, for example, comprise argon. Further, wire may moveable within the first channel. In such an embodiment, the device may further comprise means for moving the wire to a first position in which the distal end of the wire extends out of the port of the first channel and to a second position in which the distal end of said wire is within the first channel.

In another embodiment, a surgical device of the present invention comprises a body member having a proximal end and a distal end and having a first and second channels therein. The first and second channels each have a proximal end and a distal end, and the first channel has a port in a distal end region of the body member. A flexible tube is placed within the first channel and a wire is within the flexible tube. The wire has a proximal end and a distal end. A housing is connected near the proximal end of the flexible tube. The housing may comprise means for connecting the proximal end of the wire to a source of RF energy and means for connecting the proximal end of the first channel to a source of an inert ionizable gas. The means for connection may comprise any known means for connection a wire to a source of RF energy and any known means for connection a tube to a source of an inert gas. An inflatable member is connected to the body member. The inflatable member is inflatable through the second channel in the body member.

In another embodiment, a surgical device according to the present invention comprises an elongate body member having a proximal end and a distal end and having a first and second channels therein. The first and second channels each have a proximal end and a distal end. The first channel has a port in a distal end region of the tube. A wire is within the first channel. The wire has a proximal end and a distal end. A housing connected near the proximal end of the body member. The housing comprises a first connector for connecting the proximal end of the wire to a source of RF energy and a second connector for connecting the proximal end of the first channel to a source of an inert ionizable gas. An inflatable member is connected to the body member. The inflatable member is inflatable through the second channel in the body member. The housing may further comprises an adapter or adapter cable.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description, or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 illustrates a side view of a preferred embodiment of a triple channel APC dual mode therapeutic balloon dilator of the present invention.

FIG. 2 is a channel view of a preferred embodiment of a triple channel APC therapeutic balloon dilator of the present invention.

FIG. 3 illustrates a side view of a preferred embodiment of a double channel APC dual mode therapeutic balloon dilator of the present invention.

FIG. 4 is a channel view of a preferred embodiment of a double channel APC dual mode therapeutic balloon dilator of the present invention.

FIG. 5 illustrates a side view of an alternate preferred embodiment of a triple channel APC dual mode therapeutic balloon dilator of the present invention.

FIG. 6 illustrates a side view of a preferred embodiment of a triple channel APC therapeutic balloon dilator of the present invention.

FIG. 7 illustrates a side view of another preferred embodiment of a triple channel APC therapeutic balloon dilator of the present invention.

FIG. 8 illustrates a side view of a preferred embodiment of a dual channel APC therapeutic balloon dilator of the present invention.

### DETAILED DESCRIPTION OF THE PREFERRED EMBODIMENTS

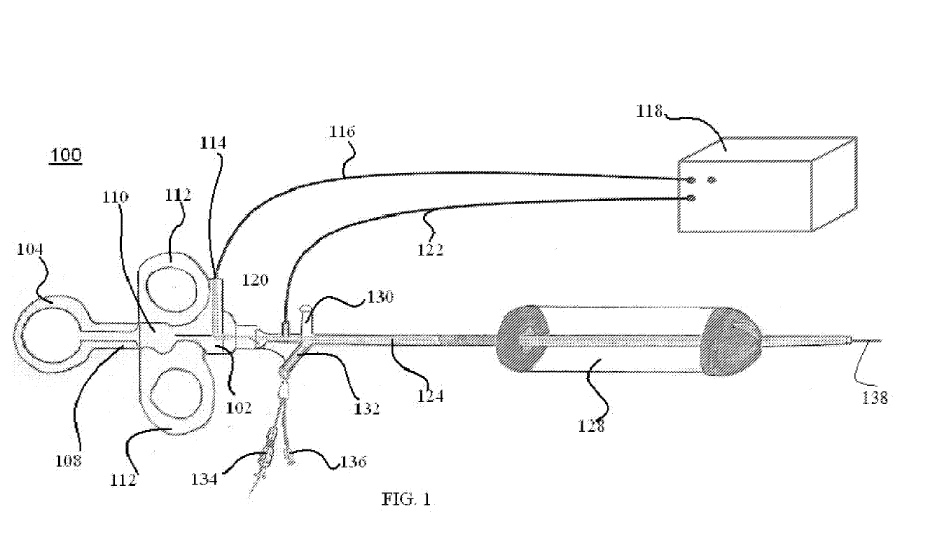

Referring to FIG. 1, a preferred embodiment of the APC dual mode therapeutic balloon dilator 100 will be described. In a preferred embodiment, a mobile ergonomic handle has a body portion 102 having a loop or circular portion 104 at one end for receiving a finger or thumb of a user. While this portion 104 of the handle is circular, rounded or a loop in this embodiment, other shapes are acceptable as well. The handle further has a slide portion 108. A manipulating portion 110 is connected to the sliding portion 108 of the handle 100. In the preferred embodiment, the manipulating portion 110 has two loops 112 for receiving fingers or a thumb of a user.

Near an end opposite the circular portion 104, the handle has a monopolar connector or connecting means 114 for connecting the device to connector cable 116 to a generator or source of RF energy 118. Although in this embodiment, the connector 114 is located at an end of the handle 100 opposite the loop 104, the connector 114 may be placed elsewhere on the handle as well.

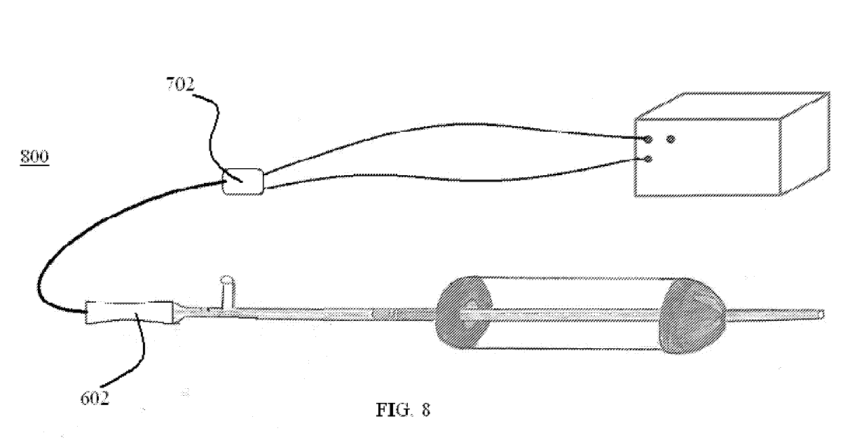

The device further has a connector 120 for connecting the device through connector line or tube 122 to a source of an inert ionizable gas such as argon, which may or not connect to the line 122 through the generator 118. Although shown as separate connectors and lines here, the monopolar connector 114 and associated line 116 and the connector 120 and line 122 may be combined in part or in whole into a single connector and line such as with a coaxial connector. Likewise, an adapter cable 702 such as is shown in FIGS. 7-8 and is known in the field may be used as well.

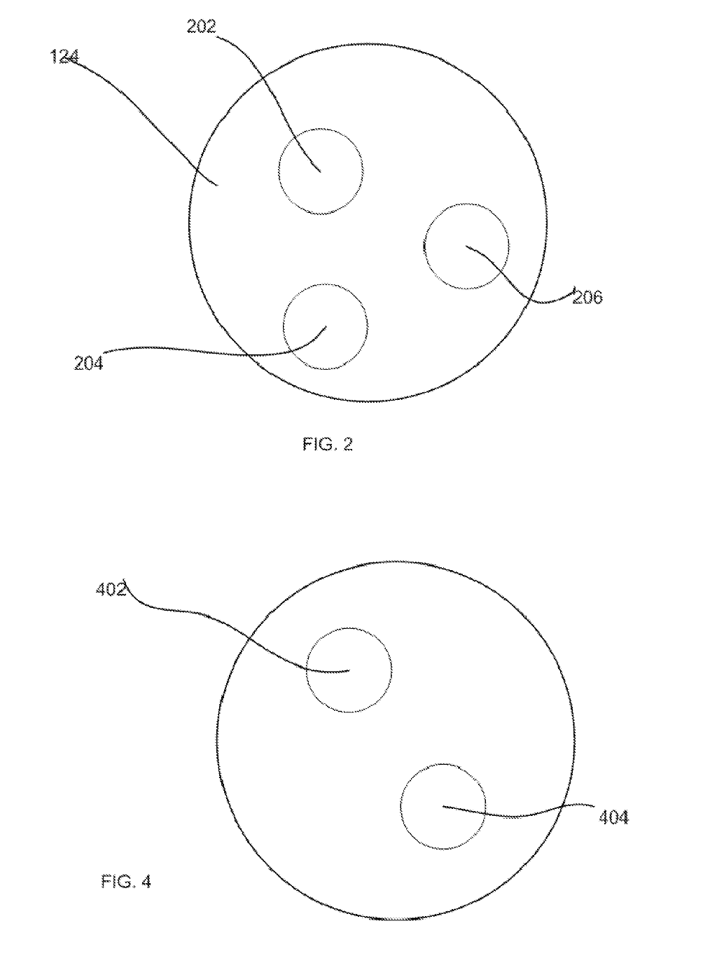

The device further has an elongate flexible body portion or catheter 124 having three channels or lumens 202, 204, 206, therein, as shown in FIG. 2. During use, the elongate flexible body portion may be inserted into an endoscope or laparoscope. Thus, the body portion 124 preferable has a diameter smaller than the diameter of the channel of a laparoscope or endoscope. For example, a typical endoscope may have a channel approximately 3.8 mm in diameter for receiving an instrument. A device in accordance with the present invention for use in such a typical laparoscope preferably would be less than 3.8 mm.

While the body portion in this embodiment has three channels, more or fewer channel may be used. The channel 202 is connected to the connector 120. A wire (not shown) extends from the handle into the proximal end of channel 202 and extends down the channel 202 toward an opening in the channel near a distal end of the body portion 124. In FIG. 2, the opening is shown at the distal end of the body portion 124, but such opening may be placed elsewhere, such as in the side of the body portion 124 near a distal end of the body portion 124. With this structure, an inert ionizable gas such as argon may be passed through the channel 202 around the wire.

On the body portion 124 is an inflatable balloon 128. In this preferred embodiment, a single elongated balloon 128 is on the body portion 124, but other embodiments having a plurality of balloon on the catheter also may be used and will be apparent to those of ordinary skill in the art.

The device further has a balloon port 130 for use in inflating the balloon 128 and a port or ports 132 for receiving other items such as a wire guide balloon 134 and/or an injection port 136 that can be inserted into channels 204, 206 in the body portion 124. In FIG. 1, a guide wire 138 is shown extending from a distal end of channel 204 or 206 in the body portion 124. The equipment or means for inflating a balloon 128 is well known in the art and therefore is not shown here.

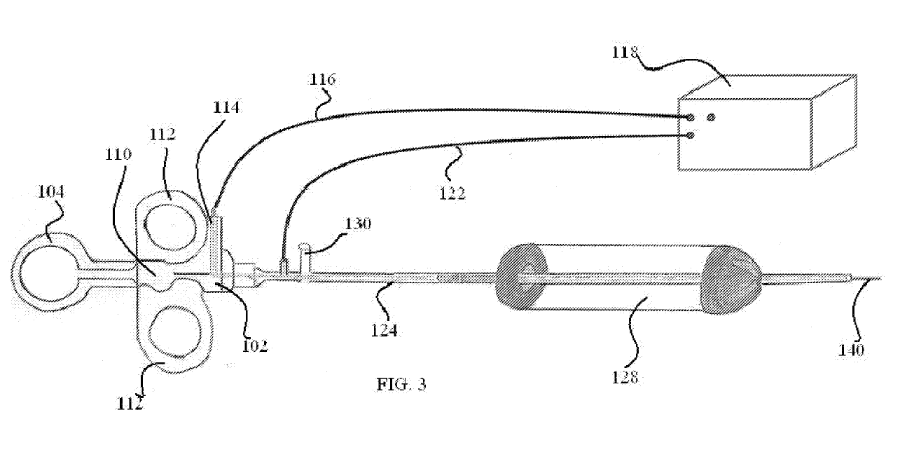

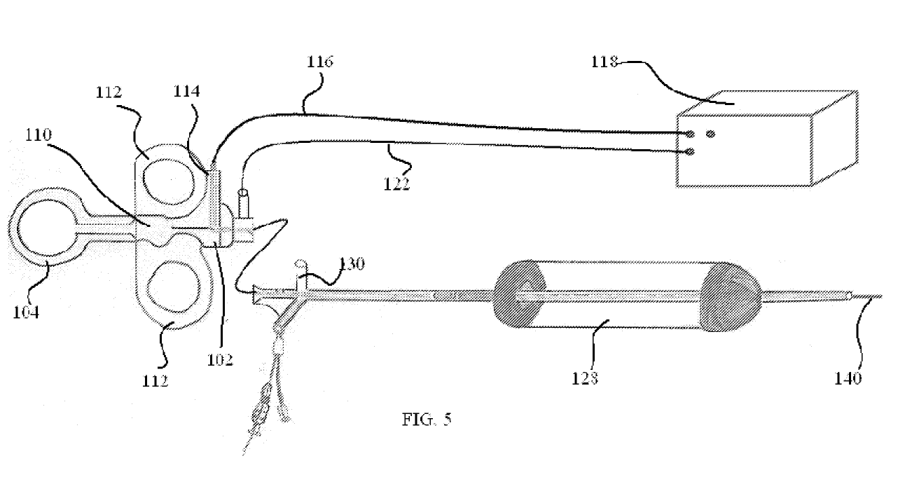

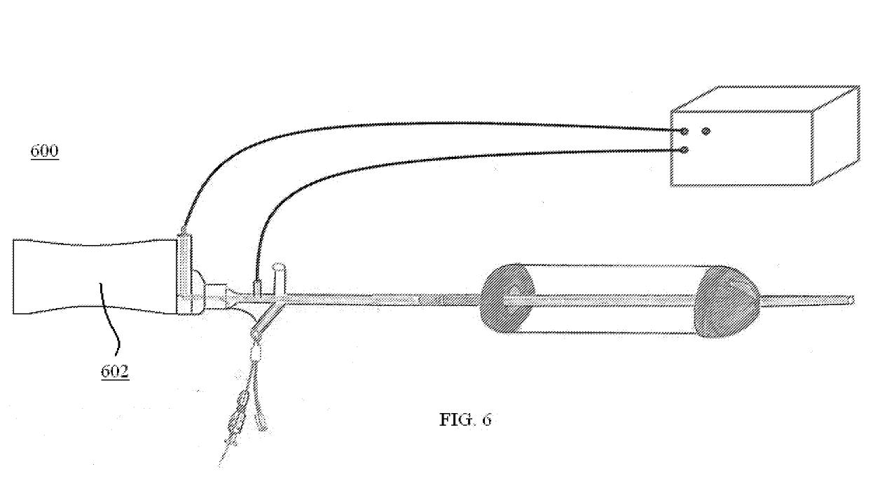

FIGS. 3-4 show a device in accordance with the present invention having only two channels 402, 404 in the body portion 124. In FIG. 3, an APC wire is shown extending from a distal end of channel 402 in the body portion 124.

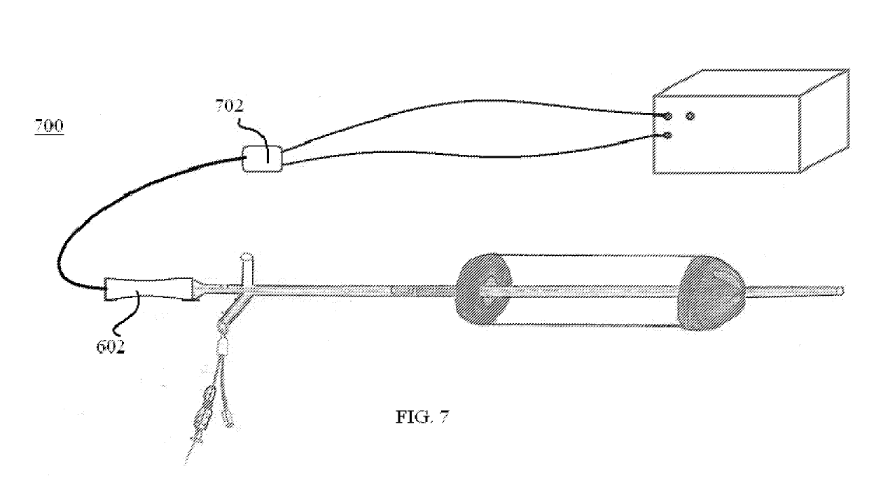

In this preferred embodiment of FIG. 3, the device includes the dual capability of performing argon beam coagulation with the wire 140 retracted into the tube 124 or performing traditional electrocautery by energizing the wire 140 while it extends from the end of the tube 124 and touching target tissue with the wire electrified. In other embodiments, such as are shown in FIGS. 6-8, the device may be modified to be a single modality device in which the wire cannot extend outside the tube 124. In such an alternative embodiment, a plug, housing or base 602 on which other components are mounted and need not be a “handle” and need not be gripped by a hand. In still other embodiments, a housing, base or plug may be used with a sliding portion or slider separate from base, housing or plug.

A device in accordance with the present invention has many uses, including, but not limited to, performing surgical procedures for removing restrictions in a colon or an esophagus. The term “surgical device” as used herein is not intended to be limiting, but rather is intended to encompass any device used to perform any medical procedure, including but not limited to being inserted percutaneously or open into intravascular or arterial vessels or inserted through any type of scope, for example an angioscope, endoscope or laparoscope.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A surgical device comprising:
an elongate catheter having a proximal end and a distal end and having a first and second channels therein, said first and second channels each having a proximal end and a distal end, and said first channel having a port in a distal end region of said elongate catheter, said elongate catheter having a diameter less than 3.8 mm;
a wire within said first channel, said wire having a proximal end and a distal end;
a housing connected near said proximal end of said tube, said housing comprising means for connecting said proximal end of said wire to a source of RF energy and means for connecting said proximal end of said first channel to a source of an inert ionizable gas; and
an inflatable member connected to said elongate catheter; said inflatable member being inflatable through said second channel in said elongate catheter.

2. A surgical device according to claim 1 wherein said elongate catheter has a third channel therein.

3. A surgical device according to claim 1 wherein said catheter is flexible.

4. A surgical device according to claim 1 wherein said catheter is rigid.

5. A surgical device according to claim 1 wherein said catheter is semi-rigid.

6. A surgical device according to claim 1 wherein said inert ionizable gas comprises argon.

7. A surgical device according to claim 1 wherein said wire is moveable within said first channel.

8. A surgical device according to claim 1 further comprising:
means for moving said wire to a first position in which said distal end of said wire extends out of said port of said first channel and to a second position in which said distal end of said wire is within said first channel.

9. A surgical device comprising:
a body member having a proximal end and a distal end and having first and second channels therein, said first and second channels each having a proximal end and a distal end, and said first channel having a port in a distal end region of said body member, said body member having a diameter less than 3.8 mm;
a flexible tube within said first channel and a wire within said flexible tube, said wire having a proximal end and a distal end;
a housing connected near said proximal end of said flexible tube, said housing comprising means for connecting said proximal end of said wire to a source of RF energy and means for connecting said proximal end of said flexible tube to a source of an inert ionizable gas; and
an inflatable member connected to said body member; said inflatable member being inflatable through said second channel in said body member.

10. A surgical device according to claim 9 wherein said body member is flexible.

11. A surgical device according to claim 9 wherein said body member is rigid.

12. A surgical device according to claim 9 wherein said body member is semi-rigid.

13. A surgical device comprising:
an elongate body member having a proximal end and a distal end and having a first and second channels therein, said first and second channels each having a proximal end and a distal end, and said first channel having a port in a distal end region of said body member, said body member having a diameter less than 3.8 mm;
a wire within said first channel, said wire having a proximal end and a distal end;
a housing connected near said proximal end of said body member, said housing comprising a first connector for connecting said proximal end of said wire to a source of RF energy and a second connector for connecting said proximal end of said first channel to a source of an inert ionizable gas; and
an inflatable member connected to said body member; said inflatable member being inflatable through said second channel in said body member.

14. A surgical device according to claim 13 wherein said housing further comprises an adapter.
